(19) United States
(12) Patent Application Publication
(10) Pub. No.: US 2005/0249795 A1
(43) Pub. Date: Nov. 10, 2005

(54) Gemcitabine compositions for better drug delivery

(75) Inventors: Jia-Ai Zhang, Vernon Hills, IL (US); Imran Ahmad, Wadsworth, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: NEOPHARM, INC., Lake Forest, IL (US)

(21) Appl. No.: 11/062,654

(22) Filed: Feb. 22, 2005

Related U.S. Application Data
(63) Continuation-in-part of application No. PCT/US03/25293, filed on Aug. 13, 2003.
(60) Provisional application No. 60/405,378, filed on Aug. 23, 2002.

(51) Int. Cl. A61K 31/7072; A61K 9/127

(52) U.S. Cl. 424/450; 514/49

(57) ABSTRACT

The present invention is for novel compositions and methods for treating cancer, particularly, for treating cancer in mammals and more particularly in humans. The therapeutic compositions of the present invention include liposome entrapped gemcitabine in which the liposome can contain any of a variety of neutral or charged liposome-forming compounds including cardiolipin.

### CROSS REFERENCE TO RELATED PATENT APPLICATIONS

This application is a continuation-in-part of PCT/US03/25293 filed on Aug. 13, 2003, which claims priority to U.S. Provisional Application No. 60/405,378 filed on Aug. 23, 2002. The disclosures of these applications are incorporated herein in their entireties by reference thereto.

### FIELD OF THE INVENTION

This invention pertains to formulations and methods for making and using gemcitabine-containing liposomes.

### BACKGROUND OF THE INVENTION

Gemcitabine is a nucleoside analogue that exhibits antitumor activity. Gemcitabine exhibits cell phase specificity, primarily killing cells undergoing DNA synthesis (S-phase) and also blocking the progression of cells through the G₁/S-phase boundary. Gemcitabine is metabolized intracellularly by nucleoside kinases to the active diphosphate (dFdCDP) and triphosphate (dFdCTP) nucleosides. The cytotoxic effect of gemcitabine is attributed to a combination of two actions of the diphosphate and the triphosphate nucleosides, which leads to inhibition of DNA synthesis. First, gemcitabine diphosphate inhibits ribonucleotide reductase, which is responsible for catalyzing the reactions that generate the deoxynucleoside triphosphates for DNA synthesis. Inhibition of this enzyme by the diphosphate nucleoside causes a reduction in the concentrations of deoxynucleotides, including dCTP. Second, gemcitabine triphosphate competes with dCTP for incorporation into DNA. The reduction in the intracellular concentration of dCTP (by the action of the diphosphate) enhances the incorporation of gemcitabine triphosphate into DNA (self-potentiation). After the gemcitabine nucleotide is incorporated into DNA, only one additional nucleotide is added to the growing DNA strands. After this addition, there is inhibition of further DNA synthesis. DNA polymerase epsilon is unable to remove the gemcitabine nucleotide and repair the growing DNA strands (masked chain termination). In CEM T lymphoblastoid cells, gemcitabine induces internucleosomal DNA fragmentation, one of the characeristics of programmed cell death.

The U.S. Food and Drug Administration (FDA) first approved gemcitabine hydrochloride for sale in the United States in 1996 as an injectable formulation under the tradename Gemzar®. The clinical formulation is supplied in a sterile form for intravenous use only. Vials of Gemzar® contain either 200 mg or 1 g of gemcitabine HCl (expressed as free base) formulated with mannitol (200 mg or 1 g, respectively) and sodium acetate (12.5 mg or 62.5 mg, respectively) as a sterile lyophilized powder. Hydrochloric acid and/or sodium hydroxide may have been added for pH adjustment.

Gemcitabine demonstrates dose-dependent synergistic activity with cisplatin in vitro. No effect of cisplatin on gemcitabine triphosphate accumulation or DNA double-strand breaks was observed. In vivo, gemcitabine showed activity in combination with cisplatin against the LX-1 and CALU-6 human lung xenografts, but minimal activity was seen with the NCI-H460 or NCI-H520 xenografts. Gemcitabine was synergistic with cisplatin in the Lewis lung murine xenograft. Sequential exposure to gemcitabine 4 hours before cisplatin produced the greatest interaction.

GEMZAR® is indicated as in combination with cisplatin for the first-line treatment of patients with locally advanced (Stage IIIA or IIIB) or metastatic (Stage IV) NSCLC. GEMZAR® is also available as first-line treatment of the treatment of locally advanced (nonresectable Stage II or Stage III) or metastatic pancreatic cancer (Stage IV) in patients. However, the toxicity of gemcitabine limits the dosage of drug that can be administered to patients. Gemcitabine HCL also has very short half-life in patients. The half-life and volume of distribution depends on age, gender and duration for infusion. Moreover, the development of multidrug resistance in cells exposed to gemcitabine can limit its effectiveness. Consequently, formulations of gemcitabine are needed that sufficiently prolong half-life of gemcitabine and maximize its therapeutic efficacy for example, by minimizing the multidrug resistance of treated cells and limiting its toxicity.

### SUMMARY OF THE INVENTION

The present invention provides for novel gemcitabine compositions, their preparation methods, and their use in treating proliferative diseases such as cancer, particularly in mammals, especially in humans. The compositions of the present invention include liposome-entrapped gemcitabine in which the liposome can contain any of a variety of neutral or charged liposome-forming materials and/or cardiolipin. The liposome-forming materials are amphiphilic molecules such as phosphatidylcholine (PC), cholesterol, phosphatidylglycerol (PG), phosphatidylserine (PS), and the like. The cardiolipin in the liposomes can be derived from natural sources or synthetic. Depending on their composition, the liposomes can carry net negative or positive charges or can be neutral. Preferred liposomes also contain α-tocopherol.

The term “Gemcitabine” as used herein means Gemcitabine hydrochloride, Gemcitabine free base and Gemcitabine derivatives.

The liposomal compositions can be used advantageously in conjunction with secondary therapeutic agents other than gemcitabine, including antineoplastic, antifungal, antibiotic among other active agents, particularly cisplatin, antisense oligonucleotides, oxaliplatin, paclitaxel, vinorelbine, epirubicin. The liposomes can be multilamellar vesicles, unilamellar vesicles, or their mixtures as desired. The invention specifically contemplates methods in which a therapeutically effective amount of the inventive liposomes in a pharmaceutically acceptable excipient are administered to a mammal, such as a human.

Desirably, the composition and method present one or more of the following advantages: 1) achieve a strong electrostatic interaction between lipids and gemcitabine, 2) avoidance of solubility problems, 3) high gemcitabine and liposome stability, 4) ability to administer gemcitabine as a bolus or short infusion in a high concentration, 5) prolong half-life of gemcitabine, 6) reduced gemcitabine toxicity, 7) increased therapeutic efficacy of gemcitabine, and 8) modulation of multidrug resistance in cancer cells. These and other properties and advantages of the present invention will be apparent upon reading the following detailed description and the accompanying figure.

### BRIEF DESCRIPTION OF THE FIGURE

FIG. 1 depicts data concerning the effect of the molar ratio between DOPG and gemcitabine hydrochloride on gemcitabine binding efficiency in liposomes

### DETAILED DESCRIPTION OF THE PREFERRED EMBODIMENTS

In one embodiment, the invention provides a composition including liposomal Gemcitabine and a negatively charged phosholipid (e.g., a first liposome-forming material), and the use of such a composition to treat cellular proliferative diseases. The Gemcitabine in the composition can be Gemcitabine hydrochloride, Gemcitabine free base, one or more Gemcitabine derivatives, or a mixture thereof.

While the negatively-charged phospholipids can be selected from among a variety of phospholipids having a negative charge, desirably the selection of the negatively charged phospholipids permits the Gemcitabine to become complexed with the negatively-charged phospholipids through electrostatic interaction. One preferred negatively charged phospholipid for inclusion in the formulation is cardiolipin, which can be, for example, natural cardiolipin, synthetic cardiolipin, or a mixture thereof. The cardiolipin can be or comprise a portion of the negatively-charged phospholipid within the composition, and it is desirable for all or a portion of the cardiolipin to be complexed with the Gemcitabine within the composition.

While the liposomal formulation including the Gemcitabine includes a negatively-charged phosopholipid, the liposomes within the composition can have a net negative or a net positive charge, or they can be neutral. The charge of the liposomes can be influenced, for example, by the presence of other liposome-forming material. In this respect, in addition to the negatively-charged phospholipid (e.g. a cardiolipin), the liposomes can include a second liposome-forming material, for example, one or more lipids such as phosphatidylcholine, cholesterol, α-tocopherol, phosphatidylglycerol and phosphatidyl serine. For example, at neutral pH, positively charged liposomes can be formed from a mixture of phosphatidylcholine, cholesterol and stearyl amine. Alternatively, negatively charged liposomes can be formed from phosphatidylcholine, cholesterol, and phosphatidyl serine.

The liposomes within the composition can be multilamellar vesicles, unilamellar vesicles, or a mixture thereof. Moreover, the liposomes can be of varying size or substantially uniform in size. For example the liposomes can have a size of about 1 mm or less, and more preferably are in the micron or sub-micron range. For example, the liposomes can have a diameter of about 5 μm or less, such as about 1 μm or less, or even 0.5 μm or less, such as about 0.2 μm or less or even about 0.1 μm or less.

Generally, the liposomes for use in the present invention can be formed by known techniques. For example, in one preferred technique gemcitabine is dissolved in an organic solvent with negatively charged phospholipids, such as cardiolipin (CL) and other phospholipids as desired and pharmaceutical excipients allowed forming complexes with gemcitabine. The cardiolipin/gemcitabine-containing mixture can be evaporated to form a film in order to facilitate electrostatic interaction and complex formation. Thereafter, solutions containing any additional desired additional lipophilic ingredients can be added to the film and the gemcitabine/lipids complexes dissolved or thoroughly dispersed in the solution. The solution can then be evaporated to form a second lipid film. A polar solvent such as an aqueous solvent can then be added to the lipid film and the resulting mixture vigorously homogenized to produce the present inventive liposomes. In another preferred technique, all of the lipophilic ingredients can be dissolved in a suitable solvent that can then be evaporated to form a lipophilic film. A polar solvent such as an aqueous solvent can then be added to the lipid film and the resulting mixture vigorously homogenized to produce the present inventive liposomes. In yet another alternative method, gemcitabine can be dissolved in a suitable aqueous solvent or buffers. The aqueous of gemcitabine can then be added to the lipid film and the resulting mixture vigorously homogenized to produce liposomes, emulsions and micelles, as desired.

Where the gemcitabine is dissolved in the lipid film as described above, the dosage form can be conveniently packaged in a single vial to which a suitable aqueous solution can be added to form the liposomes. Alternatively, a two vial system can be prepared in which the lipophilic ingredients are contained as a film in one vial and aqueous ingredients containing gemcitabine are provided in a second vial. The aqueous gemcitabine-containing ingredients can be transferred to the vial containing the lipid film and the liposomes formed by standard methods.

In a preferred embodiment, the liposomes, once formed, can be filtered through suitable filters to control their size distribution. Suitable filters include those that can be used to obtain the desired size range of liposomes from a filtrate. For example, the liposomes can be formed and thereafter filtered through a 5 micron filter to obtain liposomes having a diameter of about 5 microns or less. Alternatively, 1 μm, 500 nm, 100 nm or other suitable filters can be used to obtain liposomes of desired size. The present inventive liposomes are stable and can be filtered through microbial retentative filters to have a sterile pharmaceutical product.

In accordance with the invention gemcitabine is dissolved in a suitable solvent. Suitable solvents are those in which gemcitabine is soluble and which can be evaporated without leaving a pharmaceutically unacceptable residue. For example, non-polar or slightly polar solvents may be used, such as ethanol, methanol, chloroform, methylene chloride or acetone.

Any suitable negatively charged lipid and cardiolipin preparation can be used in the present invention. For example, cardiolipin can be purified from natural sources or can be chemically synthesized, such as tetramyristylcardiolipin, by such methods as are known in the art. Cardiolipin can be dissolved in a suitable solvent as described above for gemcitabine and the solutions mixed or the cardiolipin can be dissolved directly with gemcitabine.

In addition to cardiolipin or other negatively-charged phospholipid, any suitable liposome-forming material can be used in the present liposomes. Suitable liposome forming materials include synthetic, semi-synthetic (modified natural) or naturally occurring compounds having a hydrophilic portion and a hydrophobic portion. Such compounds are amphiphilic molecules and can have net positive, negative, or neutral charges. The hydrophobic portion of liposome forming compounds can include one or more nonpolar, aliphatic chains, for example, palmitoyl groups. Examples of suitable liposome-forming compounds include phospholipids, sterols, fatty acids, and the like. Preferred liposome forming compounds include cardiolipin, phosphatidylcholine (PC), cholesterol, phosphatidylglycerol (PG), phosphatidylserine (PS), and α-tocopherol. Phosphatidylethanolamine (PE), phosphatidic acid (PA), phosphatidylinositol (PI), sphingomyelin (SM), ganglioside G_{M1}, and polymer modified lipids, such as PEG modified lipids or a combination thereof also can be included.

As described above for the negatively-charged phospholipids (e.g., cardiolipin) and gemcitabine, the liposome-forming material can be dissolved in a suitable solvent, which can be a low polarity solvent such as chloroform, or a non-polar solvent, such as n-hexane. Other lipophilic ingredients can be admixed with the aforementioned ingredients, the ingredients can then be mixed with gemcitabine and the solvent evaporated to produce a homogeneous lipid film. Solvent evaporation can be by any suitable means that preserves the stability of gemcitabine and other lipophilic ingredients.

Liposomes can then be formed by adding a polar solution, preferably an aqueous solution, such as a saline solution, to the lipid film and dispersing the film by vigorous mixing. Optionally, the polar solution can contain gemcitabine. The solution can be pure water or it can contain salts, buffers, or other soluble active agents. Any method of mixing can be used provided that the chosen method induces sufficient shearing forces between the lipid film and polar solvent to strongly homogenize the mixture and form liposomes. For example, mixing can be by vortexing, magnetic stirring, and/or sonicating. Multilamellar liposomes can be formed simply by vortexing the solution. Where unilamellar liposomes are desired a sonication or filtration step is included in the process.

More generally, any suitable method of forming liposomes can be used so long as it provides liposome entrapped gemcitabine. Thus, solvent evaporation methods that do not involve formation of a dry lipid film can be used. For example, liposomes can be prepared by forming an emulsion in an aqueous and organic phase and evaporating the organic solvent. Reverse-phase evaporation, infusion procedures, and detergent dilution, can be used to produce the liposomes. The present invention is intended to encompass liposome-entrapped gemcitabine, without regard to the procedure for making the liposomes.

The preferred liposome entrapped gemcitabine compositions contains suitable amounts of gemcitabine. Suitable amounts can include from 1 to 50 wt. % gemcitabine, and more preferably 2 to 25 wt. % gemcitabine. Preferred compositions also contain cardiolipin, cholesterol, phosphatidylcholine and α-tocopherol in suitable amounts. The inventive compositions can contain any suitable amount of cardiolipin. Suitable amounts can include from 1 to 50 wt. % cardiolipin, and more preferably 2 to 25 wt. % cardiolipin. The inventive compositions can contain any suitable amount of phosphatidylcholine. Suitable amounts of phosphatidylcholine can include from 1 to 95 wt. % phosphatidylcholine, and more preferably 20 to 75 wt. % phosphatidylcholine. Preferred liposomes of the present invention also contain suitable amounts of α-tocopherol or other suitable antioxidants. Suitable amounts range from 0.001 wt. % to 10 wt. % α-tocopherol, such as, for example, 5 wt. % α-tocopherol. For reference, wt. % refers to the relative mass of each ingredient in the final composition without regard to the amount of added water.

To improve shelf-life and preserve liposome stability, the present invention provides gemcitabine liposome preparations which can be stored for extended periods of time without substantial leakage from the liposomes of internally encapsulated materials.

The present invention provides a gemcitabine liposome preparations, which can be dehydrated, stored for extended periods of time while dehydrated, and then rehydrated when and where they are to be used, without losing a substantial portion of loaded gemcitabine during the dehydration, storage and rehydration processes. To achieve these and other objects, the invention, in accordance with one of its aspects, provides gemcitabine liposome preparations which have been dehydrated in the presence of one or more protective sugars. In certain preferred embodiments of the invention, the liposomes are dehydrated with the one or more sugars being present at both the inside and outside surfaces of the liposome membranes. In other preferred embodiments, the sugars are selected from the group consisting of trehalose, maltose, lactose, sucrose, glucose, and dextran, with the most preferred sugars from a performance point of view being trehalose and sucrose. In general, disaccharide sugars have been found to work better than monosaccharide sugars, with the disaccharide sugars trehalose and sucrose being most effective. Other more complicated sugars can also be used. For example, aminoglycosides, including streptomycin and dihydrostreptomycin, have been found to protect liposomes during dehydration.

The dehydration is preferably achieved under vacuum and can take place either with or without prior freezing of the liposome preparation. The liposomes are preferably dehydrated using standard freeze-drying equipment or equivalent apparatus, that is, they are preferably dehydrated under reduced pressure. If desired, the liposomes and their surrounding medium can be frozen in liquid nitrogen before being dehydrated. Alternatively, the liposomes can also be dehydrated without prior freezing, by simply being placed under reduced pressure.

It has been found that invented liposomes having a concentration gradient across their membranes can be dehydrated in the presence of one or more sugars, stored in their dehydrated condition, subsequently rehydrated, and the concentration gradient then used to create a transmembrane potential which will load gemcitabine into the liposomes. Alternatively, the concentration gradient can be created after the liposomes have been dehydrated, stored, and rehydrated.

When the dehydrated liposomes are to be used, rehydration is accomplished by adding diluent, such as water for injection, normal saline, 5% dextrose in normal saline (D5W). The gemcitabine liposomes can be resuspended into the aqueous solution by gentle swirling of the solution. The rehydration can be performed at room temperature or at other temperatures appropriate to the composition of the liposomes and their internal contents.

The invention includes pharmaceutical preparations that in addition to the liposomal gemcitabine preparation, also include non-toxic, inert pharmaceutically suitable excipients and processes for the production of these preparations. The invention also includes pharmaceutical preparations in dosage units. This means that the preparations are in the form of individual parts, for example capsules, softgel capsules, pills, suppositories, ampoules and vials, of which the content of liposome entrapped gemcitabine corresponds to a fraction or a multiple of an individual dose. The dosage units can contain, for example, 1, 2, 3 or 4 individual doses or ½, 1/3 or ¼ of an individual dose. An individual dose preferably contains the amount of gemcitabine which is given in one administration and which usually corresponds to a whole, a half or a third or a quarter of a daily dose.

The abovementioned pharmaceutical preparations are manufactured in the usual manner according to known methods, for example by mixing liposomal gemcitabine with an excipient or excipients. By non-toxic, inert pharmaceutically suitable excipients there are to be understood solid, semi-solid or liquid diluents, fillers, solubilizers, stabilizer and formulation auxiliaries of all kinds.

The active compound or its pharmaceutical preparations administered locally, orally, parenterally, intraperitoneally and/or rectally, preferably parenterally, especially intravenously. Suitable amounts are therapeutically effective amounts that do not have excessive toxicity, as determined in empirical studies. Accordingly, any pharmaceutical preparation suitable to the desired route of administration, e.g., tablets, dragees, capsules, pills, granules, suppositories, solutions, suspensions and emulsions, pastes, ointments, gels, creams, lotions, powders and sprays, can be used. Suppositories can contain, in addition to the liposome-entrapped gemcitabine, suitable water-soluble or water-insoluble excipients. Suitable excipients are those in which the inventive liposomal entrapped gemcitabine are sufficiently stable to allow for therapeutic use, for example polyethylene glycols, certain fats, and esters or mixtures of these substances. Ointments, pastes, creams and gels can contain suitable excipients in which the liposome-entrapped gemcitabine is stable and can contain additives such as eucalyptus oil and sweeteners like saccharin.

The present invention also includes the use of the active compound according to the invention and of pharmaceutical preparations which contain the active compound according to the invention in human and veterinary medicine for the prevention, amelioration and/or cure of diseases, in particular those diseases caused by cellular proliferation, such as cancer. The composition can be used to treat cancer in any patient in need of such treatment, which is typically a mammalian patient, such as a cow, horse, pig, dog or cat. For example, dog lymphoma can be treated effectively with the present gemcitabine formulation. However, the present formulation is particularly preferred for use in the treatment of human patients, particularly for cancer and other diseases caused by cellular proliferation. Examples of cancers treatable by this invention include, but not limited to lung cancer (including, but not limited to unresectable, advanced non small cell lung cancer); breast cancer; testicular cancer; ovarian cancer; gastro intestinal cancers including colon, rectal, pancreatic, and gastric cancers, hepatocellular carcinoma; head and neck cancers; prostate cancer; renal cell carcinoma; adenocarcinoma; sarcomas; lymphomas; leukemias; and mycosis fugoides; melanoma; high grade glioma, glioblastoma and brain cancers.

The gemcitabine should preferably be present in the abovementioned pharmaceutical preparations in a concentration of about 0.1 to 50, preferably of about 0.5 to 25, percent by weight of the total mixture. Depending, in part, on the route of administration, the usual initial dose of gemcitabine is about 600-1500 mg/m². In a human, for example, preferably, about 800-1300 mg/m² is administered. However, it can be necessary to deviate from the dosages mentioned and in particular to do so as a function of the nature and body weight of the subject to be treated, the nature and the severity of the illness, the nature of the preparation and if the administration of the medicine, and the time or interval over which the administration takes place. Thus it can suffice in some cases to manage with less than the abovementioned amount of active compound while in other cases the abovementioned amount of active compound can be exceeded. However, determining an optimal dosage is within the ordinary skill of a practitioner in this field, and the particular required optimum dosage and the type of administration of the gemcitabine can be determined by one skilled in the art, by available methods.

One significant advantage of the present composition is that it provides a method of modulating multidrug resistance in cancer cells that are subjected to gemcitabine. In particular, the present liposomal compositions reduce the tendency of cancer cells subjected to chemotherapy with gemcitabine to develop resistance thereto, and reduces the tendency of treated cells of developing resistance to other therapeutic agents, such as cisplatin, vindesine, taxol, 5-fluorouracil (5-FU) or leucovorin, for example. Thus, other agents can be advantageously employed with the present treatment either in the form of a combination active with gemcitabine or by separate administration. Preferred agents other than gemcitabine include antineoplastic, antifungal, and antibiotic among other active agents; particularly cisplatin, antisense oligonucleotides (preferably an oligonucleotide antisense to raf (e.g., 5′-GTGCTCCATTGATGC-3′ (SEQ ID NO:1)), such as liposomal formulation of anti-c-raf oligonucleotides (see, e.g., U.S. Pat. No. 6,126,965 and 6,559,129), siRNA (preferably an siRNA directed to raf (e.g. c-raf)), oxaliplatin, paclitaxel, vinorelbine, epirubicin. Another advantage of the present composition is that the present liposomal compositions reduce the irritation, local tissue necrosis, and/or thrombophlebitis. By using the present liposomal compositions, the extravasation injuries is significantly reduced since the free gemcitabine is not in contact with the tissue directly.

### EXAMPLE 1

This is an example of lipid formulation according to the invention, with gemcitabine hydrochloride.

Lipids (85-500 μmole) were dissolved in organic solvent. The mixture was stirred gently and the solvents were evaporated under vacuum at 40-60° C. to form a thin dry film of lipids. Gemcitabine hydrochloride (70 μmole) was dissolved in 5 ml of 30 mM acetate buffer, pH 3.0. Liposomes were formed by adding the drug solution to the lipid film and aggressively mixing the components by votexing. The liposomes formed then were extruded through two stacked 0.2 μm and 0.1 μm pore size polycarbonate filters to reduce the particle size. The liposome mean diameter was determined using dynamic light scattering (DLS) technique with a Nicomp 380 Submicron Particle Sizer (Particle Sizing Systems, Santa Barbara, Calif.) equipped with auto dilution function. The gemcitabine binding efficiency in the liposome was determined by centrifuging an aliquot of the subject liposomes at 58,000 rpm for 2 hours at 4° C. Thereafter the drug was analyzed using high pressure liquid chromatography (HPLC). Generally the binding efficiency of gemcitabine in liposomes between 15-80% of the initial input dose.

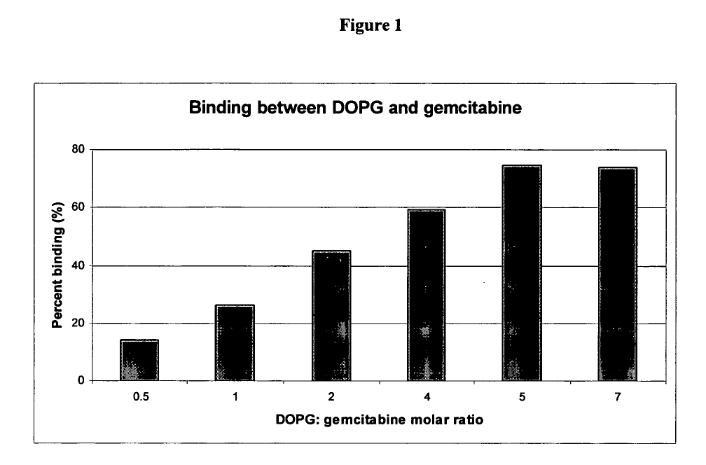

Data for several formulations are presented in Table 1, and FIG. 1 shows the effect of the molar ratio between DOPG and gemcitabine hydrochloride on the gemcitabine binding efficiency in the liposomes. Gemcitabine binding increased with an increasing molar ratio of DOPG to gemcitabine from 0.5:1 to 5:1. However the drug percent binding reached a plateau once the lipid to drug molar ratio exceeded 5:1.

TABLE 1

| | Charge | Drug binding | Vesicle size | Liposome |
|---|---|---|---|---|
| Formulation | ratio (−/+) | efficiency(%) | (nm) | formed |
| 1,1′,2,2′-Tetramyristoyl | 1.0 | 18.7 | / | yes |
| Cardiolipin | | | | |
| 1,1′,2,2′-Tetramyristoyl | 2.0 | 33.9 | / | yes |
| Cardiolipin | | | | |
| 1,1′,2,2′-Tetralauroyl Cardiolipin | 2.0 | 20.5 | 201 | yes |
| DOPG | 2.0 | 46.3 | 114 | yes |
| DOPG | 4.0 | 59.4 | / | yes |
| DOPG | 5.0 | 74.7 | 116 | yes |
| DOPG:DPPC 80:20 | 5.0 | 70.5 | 119 | yes |
| DOPG:DSPC 80:20 | 5.0 | 72.8 | 115 | yes |
| DOPG:cholesterol 80:20 | 5.0 | 67.4 | 119 | yes |
| DOPG:cholesterol sulfate 80:20 | 5.0 | 63.7 | 198 | yes |
| DOPG:chol.:cardiolipin | 5.1 | 65.0 | 116 | yes |
| 70:20:10 | | | | |
| DOPG:DSPC:cadiolipin | 5.1 | 59.8 | 107 | yes |
| 70:20:10 | | | | |
| DOPG:DSPC:DSPG 80:10:10 | 5.1 | 63.9 | 94 | yes |
| DOPG:DSPC:chol. 70:20:10 | 5.0 | 70.9 | 118 | yes |
| DOPG:DSPC:chol. 60:20:20 | 4.0 | 54.3 | 132 | yes |
| DMPG | 5.0 | 34.3 | 39.8 | yes |
| DMPG:cholesterol 80:20 | 5.0 | 33.1 | 92.3 | yes |

### EXAMPLE 2

This is an example of lipid formulation according to the invention, with gemcitabine free base.

Gemcitabine free base (76 μmole) was dissolved in organic solvent containing lipids (150-380 μmole). The mixture was stirred gently and the solvents evaporated under vacuum at 40° C. to form a thin dry film of lipids and drug. Liposomes were formed by adding 5 ml of 30 mM acetate buffer, pH 3.0 or 5 ml of 20% sucrose pH adjusted to 8.5 with NaOH and mixing the components by votexing. The liposomes formed then were extruded through two stacked 0.2 μm and 0.1 μm pore size polycarbonate filters to reduce the particle size. The liposome mean diameter was determined using dynamic light scattering (DLS) technique with a Nicomp 380 Submicron Particle Sizer (Particle Sizing Systems, Santa Barbara, Calif.) equipped with auto dilution function. The gemcitabine binding efficiency in the liposome was determined by centrifuging an aliquot of the subject liposomes at 58,000 rpm for 2 hours at 4° C. Thereafter the drug was analyzed using high pressure liquid chromatography (HPLC). Generally the binding efficiency of gemcitabine in liposomes was between 20-80 % of the initial input dose. Data for several formulations are presented in Table 2.

TABLE 2

| | | Drug | | | | |
|---|---|---|---|---|---|---|
| | | dissolved | | | Vesicle | |
| | Lipid/drug | in lipid | pH of the | Drug binding | size | Liposome |
| Formulation | ratio | film | formulation | efficiency(%) | (nm) | formed |
| DOPC:Chol.:CL | 5:1 | yes | 7.8 | 23.5 | 133 | yes |
| 50:30:20 | | | | | | |
| DOPC:Chol.:CL | 5:1 | yes | 3.8 | 33.6 | 95 | yes |
| 50:30:20 | | | | | | |
| DOPG | 2:1 | yes | 4.0 | 36.8 | 104 | yes |
| DOPG | 5:1 | yes | 4.3 | 75.2 | 105 | yes |
| DOPG | 5:1 | no | 4.0 | 48.7 | 110 | yes |

All of the references cited herein, including patents, patent applications, and publications, are hereby incorporated in their entireties by reference. While this invention has been described with an emphasis upon preferred embodiments, variations of the preferred embodiments can be used, and it is intended that the invention can be practiced otherwise than as specifically described herein. Accordingly, this invention includes all modifications encompassed within the spirit and scope of the invention as defined by the claims.

1. A liposomal composition comprising gemcitabine and a first liposome forming material, wherein the first liposome forming material comprises negatively charged phospholipids.

2. The composition of claim 1, wherein the negatively charged phospholipid is selected from a group consisting of cardiolipin, phosphatidyl serine, phosphatidic acid, phosphatidyl inositol or a mixture thereof.

3. The composition of claim 2, wherein the negatively charged phospholipids are cardiolipin.

4. The composition of claim 3, wherein the cardiolipin is selected from a group consisting of natural cardiolipin, synthetic cardiolipin or a mixture thereof.

5. The composition of claim 1, wherein the negatively charged phospholipids are pegylated.

6. The composition of claim 1, wherein the negatively charged phospholipids are linked to polyethylene glycol derivatives.

7. The composition of claim 1, further comprising a second liposome forming material.

8. The composition of claim 7, wherein the second liposome forming material comprises one or more lipids selected from a group consisting of phosphatidylcholine, cholesterol, α-tocopherol, phosphatidylglycerol, phosphatidyl serine, cationic cardiolipin or cationic cardiolipin analogs, phosphatidylethanolamine, phosphatidic acid, phosphatidylinositol, sphingomyeline, ganglioside, stearyl amine or a mixture thereof.

9. The composition of claim 8, wherein the lipids are pegylated.

10. The composition of claim 8, wherein the lipids are linked to polyethylene glycol derivatives.

11. The composition of claim 7, wherein a portion of said gemcitabine is complexed with said first and second liposome forming materials.

12. The composition of claim 11, wherein a portion of said first and second liposome forming materials interact with said gemcitabine through electrostatic interactions.

13. The composition of claim 11, wherein a portion of said first and second liposome forming materials interact with said gemcitabine through hydrophobic interactions.

14. The composition of claim 1, wherein the gemcitabine is selected from a group consisting of gemcitabine hydrochloride, gemcitabine free base, a gemcitabine derivative, or a mixture thereof.

15. The composition of claim 1, wherein said composition further comprises one or more therapeutic agents other than gemcitabine.

16. The composition of claim 15, wherein said therapeutic agent is selected from a group consisting of an antineoplastic, antifungal, or antibiotic agent.

17. The composition of claim 15, wherein said agent is selected from a group consisting of cisplatin, an anti sense oligonucleotide, siRNA, oxaliplatin, paclitaxel, vinorelbine, or epirubicin.

18. The composition of claim 17, wherein the antisense oligonucleotide is directed to raf.

19. The composition of claim 17, wherein the siRNA is directed to raf.

20. The composition of claim 1, further comprising one or more pharmaceutical acceptable excipients.

21. The composition of claim 20, wherein one or more of said excipients improves the stability of the composition.

22. The composition of claim 20, wherein at least one of said excipients is a protective sugar.

23. The composition of claim 22, wherein the sugar is selected from the group consisting of trehalose, maltose, sucrose, glucose, lactose, dextran, aminoglycoside

24. The composition of claim 1, wherein the liposome bears a negative charge.

25. The composition of claim 7, wherein the liposome bears a negative charge.

26. The composition of claim 7, wherein the liposome bears a positive charge.

27. The composition of claim 7, wherein the liposome bears a neutral charge.

28. The composition of claim 1, wherein said liposome entrapped gemcitabine comprises vesicles having a size of about 5 μm or less.

29. The composition of claim 1, wherein said liposome entrapped gemcitabine comprises vesicles having a size of about 1 μm or less.

30. The composition of claim 1, wherein said liposome entrapped gemcitabine comprises vesicles having a size of about 0.5 μm or less.

31. The composition of claim 1, wherein said liposome entrapped gemcitabine comprises vesicles having a size of about 0.1 μm or less.

32. The composition of claim 7, wherein said liposome entrapped gemcitabine comprises vesicles having a size of about 5 μm or less.

33. The composition of claim 7, wherein said liposome entrapped gemcitabine comprises vesicles having a size of about 1 μm or less.

34. The composition of claim 7, wherein said liposome entrapped gemcitabine comprises vesicles having a size of about 0.5 μm or less.

35. The composition of claim 7, wherein said liposome entrapped gemcitabine comprises vesicles having a size of about 0.1 μm or less.

36. The composition of claim 1, wherein said composition comprises a mixture of multilamellar vesicles and unilamellar vesicles.

37. The composition of claim 1, wherein said composition comprises multilamellar vesicles.

38. The composition of claim 1, wherein said composition comprises unilamellar vesicles.

39. The composition of claim 7, wherein said composition comprises a mixture of multilamellar vesicles and unilamellar vesicles.

40. The composition of claim 7, wherein said composition comprises multilamellar vesicles.

41. The composition of claim 7, wherein said composition comprises unilamellar vesicles.

42. A method of treating a cellular proliferative disease, comprising administering to a patient in need of such treatment a pharmaceutical composition comprising a therapeutically effective amount of gemcitabine encapsulated liposomes.

43. A method of modulating multidrug resistance in cancer cells, comprising administering a pharmaceutical composition comprising a therapeutically effective number of liposomes comprising gemcitabine.
